(19) United States
(12) Patent Application Publication
(10) Pub. No.: US 2021/0052822 A1
(43) Pub. Date: Feb. 25, 2021

(54) GLUCOSE RESPONSIVE COMPOSITE GEL COMPOSITION, METHOD FOR PRODUCING SAME, INSULIN DELIVERY MICRONEEDLE INCLUDING SAID GLUCOSE RESPONSIVE COMPOSITE GEL COMPOSITION, AND PRODUCTION METHOD THEREOF

(71) Applicants: National University Corporation Tokyo Medical and Dental University, Tokyo (JP); Kanagawa institute of Industrial Science and Technology, Ebina-shi (JP)

(72) Inventors: Akira MATSUMOTO, Tokyo (JP); Slyuan CHEN, Kanagawa (JP)

(21) Appl. No.: 16/982,417

(22) PCT Filed: Mar. 22, 2019
(86) PCT No.: PCT/JP2019/012026

§ 371 Date: Sep. 18, 2020

(30) Foreign Application Priority Data
Mar. 22, 2018 (JP) 2018-053817

(51) Int. Cl. A61M 5/32; A61K 9/00; A61K 47/32; A61K 47/42; A61K 38/28

(52) U.S. Cl. CPC A61M 5/32; A61K 9/0021; A61M 2207/00; A61K 47/42; A61K 38/28; A61K 47/32

(57) ABSTRACT

A gel composition which can be used suitably for a microneedle that can release insulin according to the glucose concentration in a self-regulated manner and a microneedle using the same. An insulin delivery microneedle comprises a base part fabricated with silk fibroin, at least one needle part integrally provided on the base part, and an insulin reservoir. At least a tip portion of the needle part comprises a composite gel composition comprising a copolymer comprising a phenylboronic acid-based monomer unit and silk fibroin.

### TECHNICAL FIELD

The present invention relates to an insulin delivery microneedle, more specifically, an insulin delivery microneedle (microneedle-type artificial pancreas device) that can regulate the amount of insulin delivery according to the blood glucose concentration.

### BACKGROUND ART

The blood glucose concentration (blood sugar level) is regulated within a certain range by actions of various hormones including insulin. If this regulatory function collapses, the blood sugar level increases abnormally, leading to diabetes mellitus. Treatment of diabetes mellitus usually involves measurement of blood glucose concentrations and injection of insulin. However, overdose of insulin may cause brain damage. It is therefore critical in treatment of diabetes mellitus to regulate the amount of insulin delivery according to the blood glucose concentration.

By the way, phenylboronic acid (PBA), which can bind to glucose reversibly, is highly effective in detection of glucose and self-regulated insulin delivery, and development of an insulin delivery device utilizing this property of phenylboronic acid is under way. For example, Patent Literature 1
(Japanese Patent Laid-Open No. 2016-209372) discloses an insulin delivery device having: a gel filling unit comprising a copolymer gel composition comprising phenylboronic acid-based monomers as monomers; an aqueous insulin solution filling unit surrounded by the gel filling unit; and a catheter or a needle housing the gel filling unit and having an opening part for releasing insulin.

According to the insulin delivery device disclosed in Patent Literature 1, the gel filling unit is inserted into the blood vessel in a state where it is housed inside the catheter or the needle. If the blood glucose concentration increases in this state, the gel composition in the gel filling unit binds to glucose and swells, and insulin diffused in the gel filling unit is released into blood through the opening part of the catheter or the needle. If the glucose concentration is low, the gel composition contracts, and the insulin release is reduced. This enables insulin delivery according to the glucose concentration.

However, because the delivery device described in Patent Literature 1 delivers insulin into the body through a catheter or a needle inserted into the blood vessel, it is the same as a conventional insulin injection in that insertion of the catheter or the needle causes pain. A microneedle is known as a drug delivery device not causing pain. A microneedle is a drug delivery device having many minute needles containing a drug and can deliver the drug transdermally in a noninvasive manner.

For example, Patent Literature 2 (Japanese Translation of PCT International Application Publication No. 2014-501547) discloses a microneedle-type drug delivery device having a base material part and a plurality of needle parts protruded from the base material part. In the drug delivery device disclosed in Patent Literature 2, at least the needle parts out of the base material part and the needle parts contain silk fibroin. Silk fibroin is preferred as a material for a microneedle because of the excellent biocompatibility and moderate biodegradability thereof.

Additionally, Patent Literature 3 (Japanese Translation of PCT International Application Publication No. 2017-514646) also discloses a drug delivery device having a microneedle fabricated with silk fibroin as a raw material. In the drug delivery device described in Patent Literature 3, the microneedle comprises silk fibroin, a small molecule swelling agent, and a carried drug and swells once brought into contact with the extracellular matrix, resulting in formation of a drug release route and gradual release of the drug. The drug is continuously released because the drug is stored in the silk fibroin membrane in the base part.

### CITATION LIST

### Patent Literature

Patent Literature 1: Japanese Patent Laid-Open No. 2016-209372
Patent Literature 2: Japanese Translation of PCT International Application Publication No. 2014-501547
Patent Literature 3: Japanese Translation of PCT International Application Publication No. 2017-514646

### SUMMARY OF INVENTION

### Technical Problem

Although the delivery devices described in Patent Literatures 2 and 3 have microneedles and do not cause pain, insulin cannot be delivered according to the glucose concentration in a self-regulated manner. Accordingly, to provide a device that can deliver insulin according to the glucose concentration and does not cause pain, replacing the catheter or the needle in the invention described in Patent Literature 1 with the microneedles described in Cited Documents 2 and 3 is considered.

However, because the needle part of a microneedle is minute compared with a catheter or the like, forming an opening part for releasing insulin in the needle part is difficult. Additionally, even if an opening part is formed, insulin may not be released effectively because the opening part is minute.

The object of the present invention is to provide a gel composition which can be suitably used for a microneedle that can release insulin according to the glucose concentration in a self-regulated manner, a microneedle using the same, and methods for producing the gel composition and the microneedle.

### Solution to Problem

The glucose-responsive composite gel composition of the present invention comprises a copolymer comprising a phenylboronic acid-based monomer unit and silk fibroin.

The method for producing the glucose-responsive composite gel composition of the present invention comprises a step of providing a monomer mixture comprising a phenylboronic acid-based monomer, and

a step of copolymerizing the monomer mixture in the presence of silk fibroin.

The delivery microneedle of the present invention comprises a base part fabricated with silk fibroin,

at least one needle part integrally provided on the base part, and

an insulin reservoir,

at least a tip portion of the needle part comprising the above-described glucose-responsive composite gel composition of the present invention.

The method for producing the insulin delivery microneedle of the present invention is a method for producing an insulin delivery microneedle comprising a base part and at least one needle part integrally provided on the base part, the method comprising: the steps of

providing a mold in which a cavity corresponding to the base part and the needle part is formed;

injecting a pregel solution containing a monomer mixture comprising a phenylboronic acid-based monomer and silk fibroin into a cavity portion corresponding to the needle part in the mold;

polymerizing the monomer mixture in the pregel solution to form a composite gel composition containing the silk fibroin;

injecting a silk fibroin solution into a cavity portion corresponding to the base part in the mold containing the composite gel composition;

drying the injected silk fibroin solution; and

removing the obtained molded body from the mold after the silk fibroin solution is dried.

### Advantageous Effects of Invention

According to the present invention, a glucose-responsive composite gel composition that can be suitably used for a microneedle releasing insulin according to the glucose concentration in a self-regulated manner and a microneedle using the same can be provided.

### BRIEF DESCRIPTION OF DRAWINGS

FIG. 1 is a schematic cross-sectional view showing one embodiment of the insulin delivery microneedle according to the present invention.

FIG. 2A illustrates the concept of insulin release by the insulin delivery microneedle shown in FIG. 1, showing a state of a high glucose concentration.

FIG. 2B illustrates the concept of insulin release by insulin delivery microneedle shown in FIG. 1, showing a state of a low glucose concentration.

FIG. 3 shows an SEM image of a gel composition not containing silk fibroin.

FIG. 3A schematically shows the structure of the gel composition shown in FIG. 3.

FIG. 4 shows an SEM image of a hybrid gel composition containing silk fibroin.

FIG. 4A schematically shows the structure of the gel composition shown in FIG. 4.

FIG. 5 shows an SEM image of a hybrid gel composition obtained by using an aqueous methanol solution as a solvent.

FIG. 5A schematically shows the structure of the gel composition shown in FIG. 5.

FIG. 6A shows an SEM image of a hybrid gel when silk fibroin is contained in a silk fibroin-containing pregel solution at a volume percent of 50%.

FIG. 6B shows an SEM image of a hybrid gel when silk fibroin is contained in a silk fibroin-containing pregel solution at a volume percent of 67%.

FIG. 6C shows an SEM image of a hybrid gel when silk fibroin is contained in a silk fibroin-containing pregel solution at a volume percent of 75%.

FIG. 6D shows an SEM image of a hybrid gel when silk fibroin is contained in a silk fibroin-containing pregel solution at a volume percent of 80%.

FIG. 7 is a schematic cross-sectional view showing one example of a mold used for molding a microneedle.

FIG. 8A is a graph showing a relationship between temperature and change in the volume of an NIPAAm/FPBA gel composition (monomer concentration: 1.5 mol/L) at various glucose concentrations.

FIG. 8B is a graph showing a relationship between temperature and change in the volume of an NIPAAm/FPBA gel composition (monomer concentration: 1 mol/L) at various glucose concentrations.

FIG. 8C is a graph showing a relationship between temperature and change in the volume of an SF-containing and methanol-treated semi-interpenetrating network gel composition (monomer concentration: 1.5 mol/L) at various glucose concentrations.

FIG. 8D is a graph showing a relationship between temperature and change in the volume of an SF-containing and methanol-treated semi-interpenetrating network gel composition (monomer concentration: 1 mol/L) at various glucose concentrations.

FIG. 9A is a cross-sectional SEM image of an NIPAAm/FPBA gel composition.

FIG. 9B is a cross-sectional SEM image of an SF-containing and methanol-treated semi-interpenetrating network gel composition.

FIG. 10 is a schematic cross-sectional view showing another embodiment of an insulin delivery microneedle.

FIG. 10A is a schematic cross-sectional view showing yet another embodiment of an insulin delivery microneedle.

FIG. 11 is a graph showing change in the blood sugar level with time in an in vivo evaluation of a microneedle using mice in a PBS group and a Humulin group.

FIG. 12 is a graph showing serum Humulin concentrations 30 minutes after injection of glucose in the PBS group and the Humulin group in the evaluation shown in FIG. 11.

### DESCRIPTION OF EMBODIMENTS

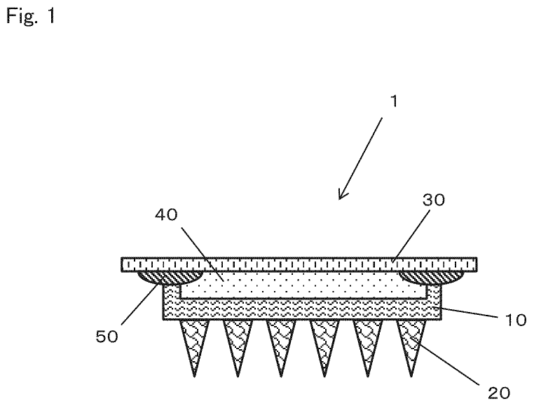

FIG. 1 is a schematic cross-sectional view showing, as one embodiment of the present invention, an insulin delivery microneedle 1 comprising a base part 10, a plurality of needle parts 20, and an insulin reservoir 40.

The needle part 20 is a portion having a sharp tip to be used to puncture the skin and is integrally provided on the base part 10. The base part 10 is a sheet-like portion supporting a plurality of needle parts 20 and has a mechanical strength that can support the needle parts 20 as well as flexibility enough to be reshaped along the skin. Additionally, for example, by forming the base part 10 in a recessed shape (cup shape), this recessed portion can be used as the reservoir 40. Insulin filled in the reservoir 40 is released outside from the surface of the needle part 20 through the base part 10 and the needle part 20.

The base part 10, the needle part 20, and the reservoir 40 that constitute the insulin delivery microneedle 1 are described in detail below.

### [Base Part]

### (Shape)

The insulin delivery microneedle 1 of this embodiment can be used as a patch attached to the skin surface. It is therefore preferred to form the base part 10 in a sheet-like shape. The planar shape of the base part 10 formed in a sheet-like shape may be an arbitrary shape such as a circular or polygonal shape and can be a rectangular shape, for example.

### (Components)

The base part 10 can be fabricated with various materials having a mechanical strength required to support the needle part 20, so that the skin can be favorably punctured with the needle part 20 against elasticity of the skin when the skin is punctured with the needle part 20, as well as insulin permeability. Examples of such materials include polymer materials and ceramics and metals that have a porous structure. Additionally, in particular, given that the base part 10 is attached to the skin surface when used, and the needle part 20 contains silk fibroin (hereinafter referred to as “SF” in the present specification) as described later, it is preferred that the base part 10 has biocompatibility or that, in addition thereto, the base part 10 is further fabricated with a material that does not obstruct continuity with the needle part 20.

Given the above, it is more preferred to fabricate the base part 10 with SF. By fabricating the base part 10 with SF, the base part 10 can be constituted so that biocompatibility is further imparted to the base part 10 having a required mechanical strength and insulin permeability, and continuity with the needle part 20 is favorably maintained without a dissimilar interface with the needle part 20.

When the base part 10 is fabricated with SF, the base part 10 can be formed by adding a solvent to purified SF appropriately, injecting the obtained SF solution into a mold, and drying it. Commercially available products can be used as purified SF itself. Because the SF solution can be prepared by a known method, explanation of preparation thereof is omitted here.

### [Needle Part]

### (Shape, Disposition, Etc.)

The length of the needle part 20 is preferably 5 mm or shorter, more preferably 1 mm or shorter as long as the needle is long enough to reach the horny layer when the needle part 20 punctures the skin. The number and the disposition of the needle parts 20 may be arbitrary. For example, a plurality of needle parts 20 can be positioned in a matrix of M×N (M and N each are an integer of 10 to 30). As one specific example of the disposition, 10×12 needle parts 20 are positioned at pitches of 500 μm in a rectangular region of 8 mm×8 mm. The shape of the needle part 20 may be arbitrary as long as the needle has a tip with which the skin can be punctured, and the shape can be preferably pyramidal.

### (Components)

At least a tip portion of the needle part 20 comprises a composite gel composition comprising a copolymer comprising a phenylboronic acid-based monomer unit and SF. The composite gel composition is obtained specifically by copolymerizing a monomer mixture comprising phenylboronic acid-based monomers in the presence of SF as described later, resulting in formation of a composite gel in which SF molecules are virtually uniformly dispersed and distributed in a crosslinked molecular structure of a copolymer. In the present application, the term “monomer unit” refers to a structural unit in a (co)polymer made up of monomers, and the term “monomer” may be used with a meaning of a “monomer unit” in descriptions below. A phenylboronic acid-based monomer refers to a monomer having a phenylboronic acid functional group represented by the following formula:

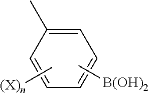

wherein X represents a substituent group, preferably F, and n is an integer of 1 to 4.

### <Gel Composition>

The present invention utilizes such a mechanism that a phenylboronic acid structure changes the structure thereof according to the glucose concentration as described below.

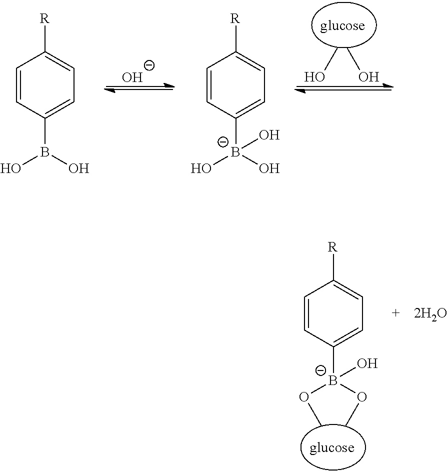

Phenylboronic acid (hereinafter sometimes referred to as “PBA” in the present specification) dissociated in water binds to a sugar molecule reversibly and maintains the equilibrium state shown above. When the glucose concentration increases, the volume of the PBA structure also increases because of the binding to glucose, and when the glucose concentration is low, the volume decreases. This reaction occurs at a gel interface brought into contact with blood in a state where the skin is being punctured with the needle part 20, and the gel contracts only at the interface, resulting in generation of a dehydrated shrink layer referred to as a “skin layer” by the present inventors. The present invention utilizes this property to regulate the release of insulin.

A gel composition that can be suitably used is a gel composition comprising a copolymer comprising a phenylboronic acid-based monomer unit having the above-described property, and the gel composition of the present invention is a composite gel composition in which SF is dispersed and compounded in this gel composition. Examples of a gel composition not containing SF are not particularly limited but include the gel composition described in Japanese Patent No. 5696961.

The phenylboronic acid-based monomer used for the preparation of a gel composition is not limited but is represented by, for example, the following general formula:

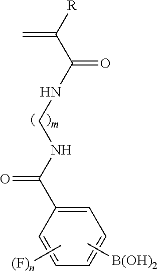

wherein R is H or CH₃, F is independently present, n is any of 1, 2, 3, and 4, and m is an integer of 0 or 1 or greater.

The above-described phenylboronic acid-based monomer has a structure in which a fluorinated phenylboronic acid (hereinafter sometimes referred to as “FPBA” in the present specification) group, in which hydrogen on a phenyl ring is substituted with 1 to 4 fluorine atoms, and a carbon of amide group binds to the phenyl ring. A phenylboronic acid-based monomer having the above-described structure is highly hydrophilic, and the pKa can be set to 7.4 or lower, which is a biological level, because a phenyl ring is fluorinated. Further, this phenylboronic acid-based monomer can become a gel that can cause a phase change depending on the glucose concentration because it can not only acquire a sugar recognizing ability in the biological environment but also can be copolymerized with a gelling agent and a crosslinking agent described later with an unsaturated bond.

When one hydrogen on a phenyl ring is substituted with fluorine in the above-described phenylboronic acid-based monomer, F and B(OH)₂ may be introduced at any of the ortho, meta, and para positions.

In general, pKa can be made lower for a phenylboronic acid-based monomer in which m is 1 or greater compared with a phenylboronic acid-based monomer in which m is 0. m is, for example, 20 or smaller, preferably 10 or smaller, more preferably 4 or smaller.

One example of the above-described phenylboronic acid-based monomer is a phenylboronic acid-based monomer with n being 1 and m being 2, and this is 4-(2-acrylamidoethylcarbamoyl)-3-fluorophenylboronic acid (AmECFPBA), which is particularly preferred as a phenylboronic acid-based monomer.

As SF contained in a composite gel composition, the same SF as used for the base part 10 can be used. SF imparts a mechanical strength to the needle part 20. The amount (weight of the solid content) of SF can be determined so that the mechanical strength of a microneedle becomes a suitable value and can be determined as, for example, 10 to 90 parts by weight, preferably 24 to 60 parts by weight, more preferably 40 to 60 parts by weight based on 100 parts by weight of the total monomer (phenylboronic acid-based monomers, a gelling agent, and a crosslinking agent) weight. The mechanical strength of a composite gel composition can be increased by increasing the weight fraction of SF based on the total monomer weight. However, if the weight fraction of SF is increased, the monomer concentration is reduced accordingly. Because forming a gel composition becomes difficult if the monomer concentration is too low, it is important to determine the weight fraction of SF based on the total monomer weight within a range that would not inhibit the formation of the gel composition.

A composite gel composition can be prepared with a gelling agent having a property of not causing a toxic effect or an adverse effect on the biological function in a living body (biocompatibility), the above-described phenylboronic acid-based monomers, and a crosslinking agent. The preparation method is not particularly limited, but a composite gel composition can be prepared by mixing monomer components comprising a gelling agent, phenylboronic acid-based monomers, and a crosslinking agent, which become the principal chain of a gel (copolymer), in a predetermined charging mole ratio and an SF solution and polymerizing the monomers in the presence of SF. A polymerization initiator is used for polymerization, if necessary.

It is preferred to add insulin to the composite gel composition beforehand. To that end, insulin can be diffused in a gel by immersing the gel in an aqueous solution such as a phosphate buffer aqueous solution containing insulin at a predetermined concentration. Subsequently, a drug can be enclosed (loaded) into the needle part 20 by immersing the gel removed from the aqueous solution in, for example, hydrochloric acid for a predetermined time to form a thin dehydrated shrink layer (referred to as a skin layer) on the surface of the gel main body.

A preferred ratio of the gelling agent, the phenylboronic acid-based monomers, and the crosslinking agent varies depending on the monomers used or the like and is not particularly limited, and a composition that can regulate the release of insulin according to the glucose concentration under physiological conditions is sufficient. The present inventors already prepared gels by combining a gelling agent and a crosslinking agent with various phenylboronic acid-based monomers in various ratios and studied their behaviors (refer to Japanese Patent No. 5622188, for example). Those skilled in the art can obtain a gel with a preferable composition on the basis of the description in the present specification and technical information reported in this field.

If a gel main body formed with a copolymer obtained by using a gelling agent, phenylboronic acid-based monomers, and a crosslinking agent and SF can be swollen or contracted in response to the glucose concentration, can maintain a characteristic of pKa of 7.4 or lower, and can be formed as a gel, the gel can be prepared by setting the charging mole ratio of a gelling agent and phenylboronic acid-based monomers to a suitable value.

As a gelling agent, a biocompatible material that has bioompatibility and can be gelled is sufficient, and examples thereof include acrylamide-based monomers having biocompatibility. Specific examples include N-isopropylacrylamide (NIPAAm), N,N-dimethylacrylamide (DMAAm), and N,N-diethylacrylamide (DEAAm).

As a crosslinking agent, similarly, a substance that has biocompatibility and can be crosslinked with monomers is sufficient, and examples thereof include N,N′-methylenebisacrylamide (MBAAm), ethylene glycol dimethacrylate (EGDMA), N,N′-methylenebismethacrylamide (MBMAAm), and other various crosslinking agents.

In one preferred embodiment of the present invention, as shown below, a composite gel composition is obtained by dissolving N-isopropylmethacrylamide (NIPAAm), 4-(2-acrylamide ethylcarbamoyl)-3-fluorophenylboronic acid (AmECFPBA), N,N′-methylenebisacrylamide (MBAAm), and SF in a solvent in a suitable mixing ratio for polymerization. It is preferred to perform polymerization at normal temperature under an aqueous condition to prevent damage of SF.

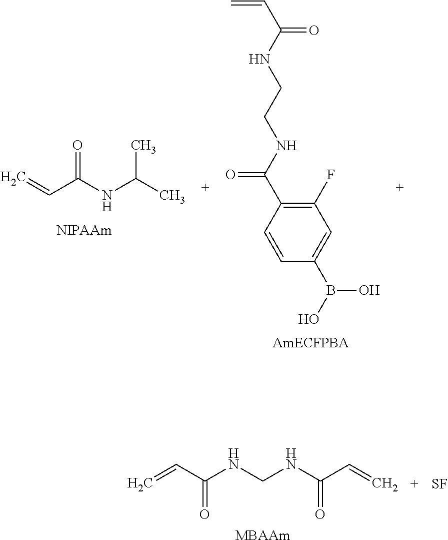

As a solvent, an arbitrary solvent that can dissolve monomers and SF can be used. Examples of such a solvent include water, alcohols, dimethyl sulfoxide (DMSO), dimethyl formamide (DMF), tetrahydrofuran (THF), ionic liquid, and combinations of one or more thereof. Of these, an aqueous methanol solution can be preferably used as a solvent.

A pregel solution is prepared by dissolving a gelling agent, PBA, a crosslinking agent, and SF in such a solvent to perform polymerization. Of note, because SF is easily gelled, it is preferred to dissolve a gelling agent, PBA, a crosslinking agent, and the like in a solvent and then add SF to the solution in a state of an SF solution when a pregel solution is prepared. In the present specification, “a pregel solution before addition of SF” and “a pregel solution after addition of SF” may be distinguished to explain a “pregel solution.”

When an aqueous methanol solution is used as a solvent, an aqueous alcohol solution with, for example, 40 volume % of methanol in a pregel solution before addition of SF can be used. In this case, the volume % of methanol in the pregel solution after addition of SF is preferably 3 to 30 volume %, more preferably 5 to 20 volume %, most preferably 8 volume %. When an aqueous ethanol solution is used as a solvent, the solubility of PBA in ethanol is low. Therefore, the volume % of ethanol in the pregel solution before addition of SF is preferably higher than the case of using an aqueous methanol solution, for example, 60 volume %.

In the present specification, a gel obtained by using a pregel solution containing a gelling agent, PBA, a crosslinking agent, and SF may be referred hereinafter to as a “hybrid gel” to distinguish from a gel obtained by using a pregel solution not containing SF. In the present specification, “gel,” “hydrogel” and “gel composition” have the same meaning, unless otherwise specified.

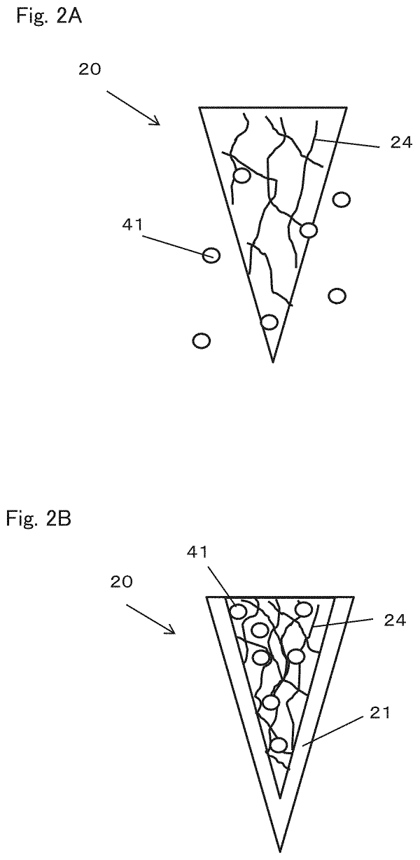

In the above-described composite gel composition, phenylboronic acid-based monomers are copolymerized with a gelling agent and a crosslinking agent to form a gel main body, and SF is uniformly distributed therein. The composite gel composition can be constituted, so that insulin is diffused in the gel, and the surface of the gel main body is surrounded by a dehydrated shrink layer. When this structure is applied to the needle part 20, for example, a gel constituting the needle part 20 with pKa of 7.44 or lower swells when the glucose concentration increases under a physiological condition at a temperature of 35° C. to 40° C. as shown in FIG. 2A. Therefore, a dehydrated shrink layer is eliminated, and the density of SF 24 decreases, and insulin 41 in the gel can be released outside.

In contrast, when the glucose concentration decreases again, as shown in FIG. 2B, the swollen gel is contracted, and a dehydrated shrink layer (skin layer) 21 is re-formed over the entire surface, the density of SF increases, and the insulin 41 in the gel can be prevented from being released outside.

Therefore, the gel composition used in the present invention can release insulin autonomically in response to the glucose concentration.

Catalysts such as an initiator and a promoting agent can be used for polymerization. As an initiator, for example, ammonium persulfate (APS) can be used. As a promoting agent, for example, tetramethylethylenediamine (TEMED) can be used. In this case, when 6.2 μL of ammonium persulfate and 12 μL of tetramethylethylenediamine per mL of a pregel solution, which correspond to 10% by weight, were polymerized at room temperature, gelation began within 10 minutes.

Various parameters affect polymerization results. Table 1 shows parameters that affect polymerization and the effects.

TABLE 1

| Parameter | Effect |
|---|---|
| Solvent | A solvent affects the degree of dissolution of a monomer, |
| | the reaction rate, and the exterior appearance of a gel. |
| | When a solvent is an aqueous methanol solution, the SF |
| | structure changes if the proportion of methanol is high, |
| | and the solubility of a PBA is deteriorated if the |
| | proportion is low. |
| APS | An APS concentration affects the rea.ction rate and the |
| concentration | exterior appearance of a gel. If the APS concentration is |
| | low, the formed gel is white and has a non-uniform |
| | structure. |
| TENTED | A TEMED concentration affects the reaction rate. |
| concentration | |
| Temperature | A (polymerization) reaction at low temperature reduces |
| | the reaction rate substantially. If the APS/TEMED |
| | concentration is lower than a threshold, gelation cannot |
| | be observed in an ice bath. |
| Monomer | A monomer concentration affects the exterior appearance |
| concentration | of a gel. The gel is transparent with a monomer |
| | concentration of lower than 1 mol/L. The mechanical |
| | strength of a gel is increased with a higher monomer |
| | concentration. |
| Percent | A percent crosslinking affects the rigidity of a gel. The |
| crosslinking | mechanical strength of a gel is increased with a higher |
| | percent crosslinking. |
| SF | An SF concentration affects the rigidity of a gel. The |
| concentration | mechanical strength of a gel is increased with a higher |
| | SF concentration. |

Taking into account these effects, a preferred combination is as follows:

Gelling agent (NIPAAm)/FPBA=92.5 mol/7.5 mol (*),
Monomer concentration=0.4 to 1.5 mol/L,
Charging percent of a crosslinking agent to the monomers=5% to 20%,
Crosslinking agent: MBAAm,
Solvent: an aqueous methanol solution; the proportion of methanol in the pregel solution before addition of SF is 40 volume % (therefore, the proportion of methanol decreases after SF is added).
(*) This ratio may vary depending on the assumed use environment or the like.

### (Structural Characteristics of a Gel Composition)

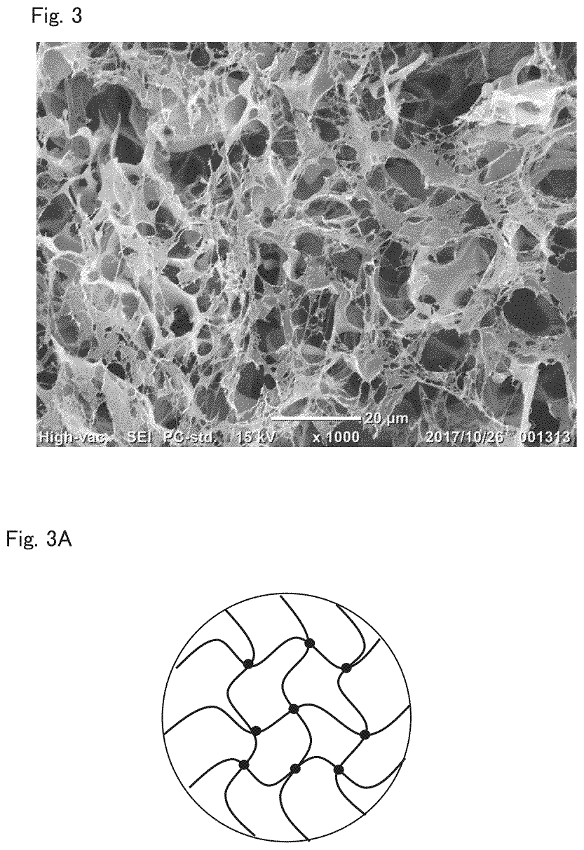

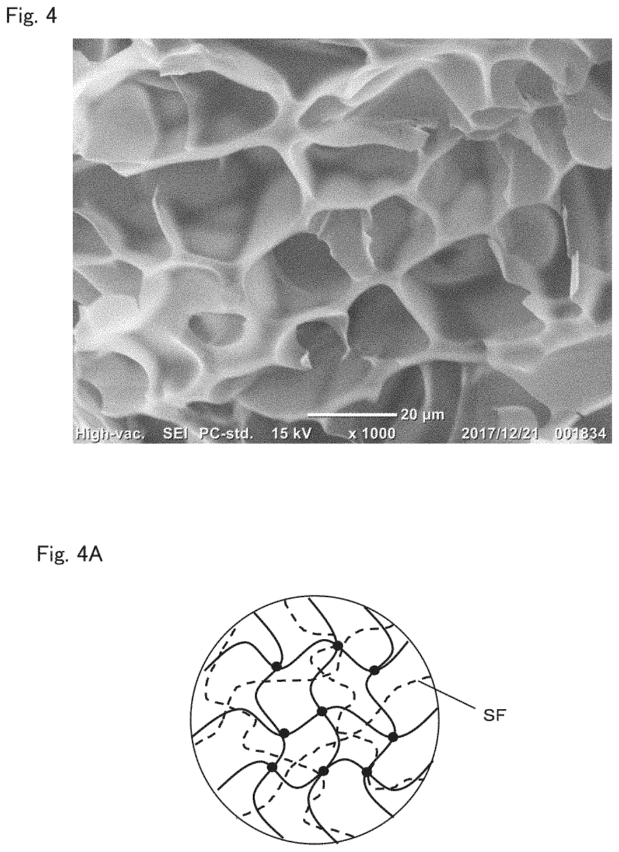

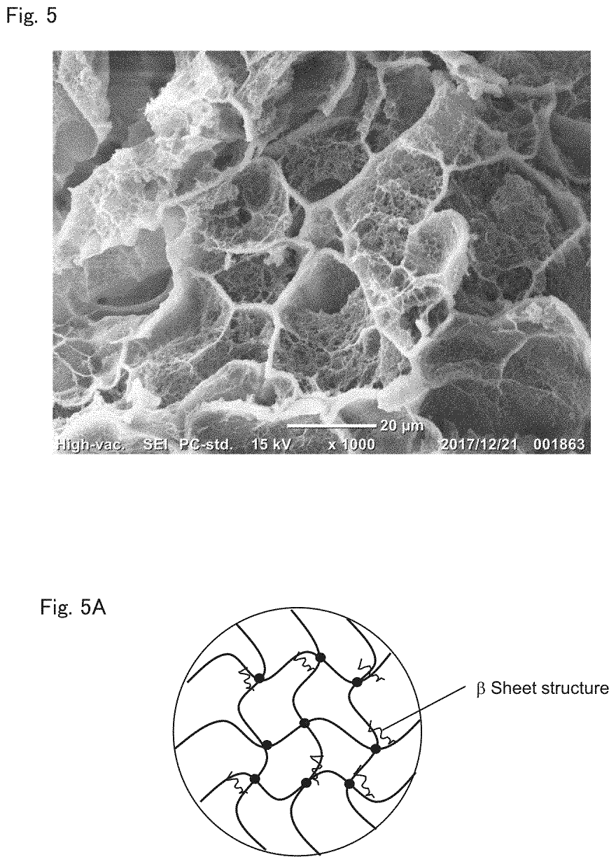

As gel compositions, scanning electron microscope (SEM) images of a gel composition not containing SF, a gel composition containing SF, and a methanol-treated gel composition are shown in FIGS. 3 to 5. An NIPAAm/PBA gel composition not containing SF is shown in FIG. 3 (Sample 1), an NIPAAm/PBA/SF hybrid gel composition containing SF is shown in FIG. 4 (Sample 2), and a hybrid gel composition using an aqueous methanol solution as a solvent is shown in FIG. 5 (Sample 3). The schematic views of structures of Samples 1 to 3 are shown in FIGS. 3A, 4A, and 5A.

Of note, Sample 1 is a gel composition obtained by using a pregel solution of the following prescription:

Gelling agent: NIPAAm,
Phenylboronic acid-based monomer: FPBA,
Crosslinking agent: MBAAm,
Solvent: aqueous methanol solution,
Volume % of methanol in a pregel solution after addition of SF=8 volume %,
NIPAAm/FPBA=92.5 mol/7.5 mol,
Charging percent of a crosslinking agent=20%,
Monomer concentration=0.6 mol/L.

Sample 2 is a gel composition obtained by using a pregel solution obtained by adding SF to the pregel solution of the Sample 1 prescription so that the weight fraction of SF based on the total monomer weight is 48% by weight. Sample 3 is a gel composition obtained by using the pregel solution of the same prescription as in Sample 2 to obtain a gel composition and then immersing the gel composition in an aqueous methanol solution containing 90 volume % of methanol for 30 minutes (methanol treatment).

FIGS. 4 and 4A show that the hybrid gel composition forms an interconnected microporous structure. This structure enables dynamic control of phase separation of two materials during polymerization and smooth and continuous release of the filled insulin. The hybrid gel composition has a greater pore size and a greater wall thickness compared with the gel composition not containing SF (FIGS. 3 and 3A). As shown in FIGS. 5 and 5A, change of the SF structure to D sheet is promoted by methanol treatment, leading to crystallization and an improved mechanical strength.

Incorporation of SF into the polymer network inhibits mobility of the polymer chain (inhibits mobility of water). Therefore, the hybrid gel composition reduces the swelling ratio, the equilibrium water content, and the sol fraction of the gel composition compared with the gel composition not containing SF. Addition of SF into the polymer network increases the wall thickness of the porous structure, resulting in reduction of the swelling ratio and the equilibrium water content. Crystallization of SF leads to a denser and more solid structure and thereby suppresses swelling of the gel composition. The swelling ratio, the equilibrium water content, and the sol fraction also depend on the percent crosslinking and the values thereof decrease as the percent crosslinking increases. It is considered that this is because the network is relaxed with a lower percent crosslinking and has a hydrodynamic free volume that houses more solvent molecules, resulting in increases in the matrix swelling, the water content, and the sol fraction.

### (Degradability of a Gel Composition)

To examine the degradability of a gel composition, the above-described Samples 1 to 3 were washed with deionized water for 2 days to remove soluble components and subsequently immersed in phosphate-buffered saline (PBS) with pH 7.4 at 37° C. After 6 days, 12.4±6.4% of Sample 1 was degraded. The percent degradation of Sample 2 was 25.1±4.2%, higher than that of Sample 1. This result indicates that SF in the hybrid gel composition is unstable without methanol treatment and can be released during incubation. However, in the methanol-treated Sample 3, the percent degradation decreases to lower than 2% after incubation at 37° C. for 6 days. The change in the SF structure due to methanol treatment not only stabilizes SF in the gel composition, but also tightens the gel composition, reducing degradation of the polymer network. These results reinforce advantages of the hybrid gel composition.

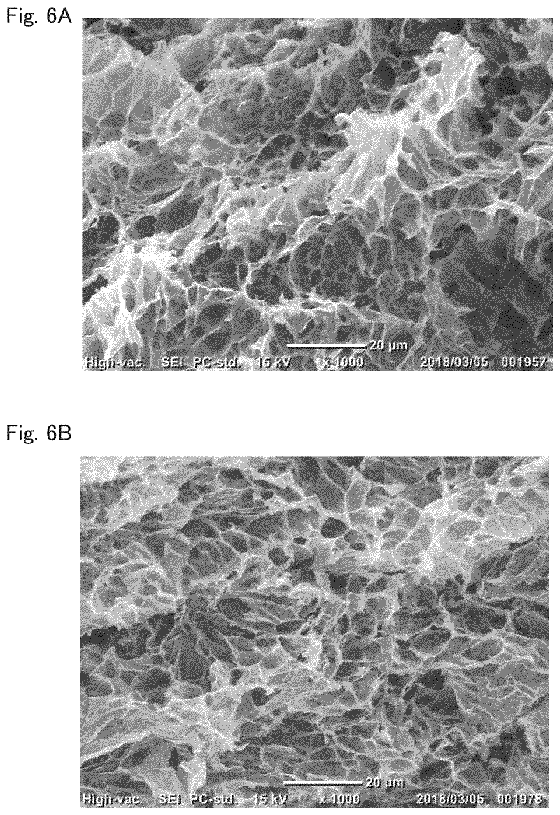

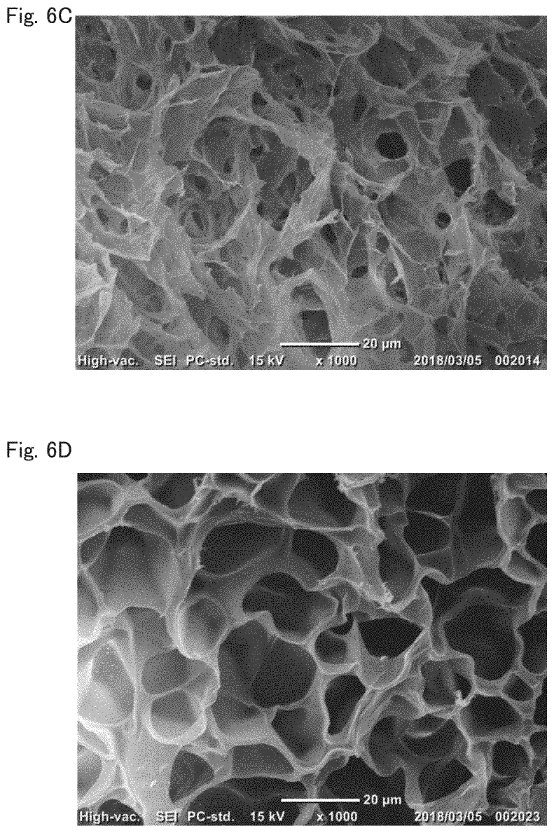

The structure of the gel composition also changes depending on the SF concentration. FIGS. 6A to 6D show SEM images of hybrid gel compositions with various SF concentrations. When the SF concentration is represented by an SF volume percent, i.e., volume of SF/volume of SF-containing pregel solution x 100(%), FIG. 6A shows an SEM image of a structure with a 50% concentration, FIG. 6B shows an SEM image of a structure with a 67% concentration, FIG. 6C shows an SEM image of a structure with a 75% concentration, and FIG. 6D shows an SEM image of a structure with an 80% concentration. These SF concentrations were 12%, 24%, 36%, and 48% as represented by the SF weight fraction in the pregel solution in FIGS. 6A, 6B, 6C, and 6D, respectively. Of note, the monomer concentration in the pregel solution was 0.6 mol/L in all the structures, and the pregel solution was transparent.

FIGS. 6A to 6D show that all hybrid gel compositions form an interconnected porous structure. Additionally, it is shown that as the SF concentration increases, the pore size and the wall thickness increase. Such a change in a structure appears favorably with an SF weight fraction of 24% or higher and appears markedly with that of 36% or higher. The change in the structure due to the increased SF concentration can contribute to the increase in the mechanical strength of the hybrid gel composition. In fact, as the SF concentration increases, the mechanical strength of the hybrid gel composition is improved.

### [Reservoir]

The reservoir 40 is important for the insulin delivery microneedle 1 to release insulin over along period (e.g., 7 days). A recessed part is formed in the base part 10 and can be utilized as the reservoir 40. In this case, a sheet covering the recessed part is bonded on the upper face of the base part 10 so that a sealed space between the base part 10 and a sheet 30 is formed as the reservoir 40. To bond the sheet 30, for example, a water-resistant adhesive agent 50 can be used. The sheet 30 is not particularly limited but, for example, a silicone sheet with a thickness of 0.3 mm can be used in light of water resistance and flexibility. Insulin can be filled into the reservoir 40 with a syringe injection through the sheet 30.

### [Formation of a Base Part and a Needle Part]

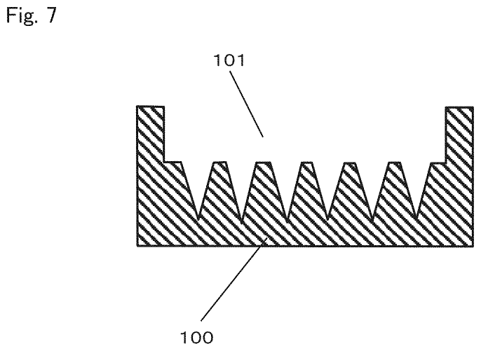

The base part 10 and the needle part 20 can be formed with a micromolding technique using a mold. Because the needle part 20 is formed integrally with the base part 10, a mold 100 having a cavity 101 formed in a shape of a needle part and a base part combined is preferred as shown in FIG. 7.

To form the base part 10 and the needle part 20 using the mold 100, first, a pregel solution obtained by dissolving materials constituting the needle part 20 in a solvent is poured into a portion corresponding to the needle part 20 in the mold 100 and polymerized to form the needle part 20. Pouring the pregel solution and polymerization thereof may be divided into multiple steps. Subsequently, an SF solution obtained by dissolving SF constituting the base part 10 in a solvent is poured into a portion corresponding to the base part 10 of the mold 100 with the needle part 20 formed therein and dried. The obtained molded body is removed from the mold 100. By doing so, the base part 10 and the needle part 20 that are integrally formed can be obtained.

Because the needle part 20 has a very minute structure, it is important to fill the pregel solution to the tip portion of the needle part 20 when the needle part 20 is formed. Examples of such a method include a centrifugation method and a vacuum molding method.

The centrifugation method is a method using a centrifuge. More specifically, the mold 100 with a pregel solution poured therein is placed in a Falcon tube and is centrifuged using a centrifuge. By this method, a pregel solution can be filled into the tip of the mold 100. Then, the needle part 20 can be formed by placing the mold 100 in a desiccator to dry the pregel solution.

In the vacuum molding method, the mold 100 is fabricated with a porous material, the mold 100 is placed under reduced pressure to remove air from the mold 100, and a pregel solution is poured into the mold 100. By doing so, the pregel solution can be filled into the tip portion of the needle part 20. As a porous material constituting the mold 100, for example, polydimethylsiloxane (PDMS) can be used.

Whether the centrifugation method or the vacuum molding method is used, no major differences are observed in the obtained shape of the needle part 20, and both the centrifugation method and the vacuum molding method can be used in the present invention.

### [Production of a Microneedle]

Several experiments were conducted to examine the method for producing the microneedle of the present invention.

### Reference Experiment 1-1

This experiment was conducted, with a fundamental concept that a needle part is formed with SF and the needle part formed with SF and a PBA gel are combined, to examine the efficacy of a first method of coating the needle part with the PBA gel.

First, an SF solution was poured into the mold 100 shown in FIG. 7. After the SF solution poured into the mold 100 was dried, a molded body fabricated with SF was removed from the mold 100 and immersed in a pregel solution for five minutes. For the pregel solution, the following Prescription 1 was used: a gelling agent (NIPMAAm)/phenylboronic acid-based monomer (FPBA)=92.5 mol/7.5 mol; pure methanol as a solvent; the monomer concentration in pure methanol=3 mol/L; percent crosslinking=5% to 20%; and azobisisobutyronitrile (AIBN) as an initiator.

The molded body immersed in the pregel solution was placed in a liquid paraffin for liquid seal. The molded body placed in the liquid paraffin was transferred to an oven at 60° C. and left overnight to polymerize the pregel solution attached on the surface of the molded body. Subsequently, the liquid paraffin was removed from the molded body by a methanol wash, and the molded body was further washed with ultrapure water and dried to obtain a microneedle.

The microneedle was formed favorably. However, magnifying observation under a microscope showed that it was difficult to determine whether the SF surface of the needle part was coated with the PBA gel.

### Reference Experiment 1-2

This experiment shares a common fundamental concept with Reference Experiment 1-1 but is different from Reference Experiment 1-1 in that a pregel solution of a different prescription was used, and the polymerization condition is therefore also different from that in Reference Experiment 1-1. In this experiment, a pregel solution (Prescription 2) different from Prescription 1 in the following points was used: ammonium persulfate (APS) was used as an initiator, and tetramethylethylenediamine (TEMED) was added as a promoting agent.

First, as in Reference Experiment 1-1, a molded body fabricated with SF was molded and removed from the mold. The obtained molded body was immersed in the pregel solution of Prescription 2 for five minutes, and then the molded body was removed from the pregel solution and left at room temperature for polymerization. After one hour, the molded body was washed with ultrapure water and dried to obtain a microneedle.

In this experiment, rapid gelation was achieved at room temperature. However, magnifying observation under a microscope showed that the surface of the needle part was not uniformly coated with the gel.

### Reference Experiment 1-3

In this experiment, a microneedle was obtained by the same procedure as in Reference Experiment 1-2 except that a pregel solution of Prescription 3, which is different from the pregel solution of Prescription 2 used in Reference Experiment 1-2, was used. The pregel solution of Prescription 3 is different from the pregel solution of Prescription 2 only in that an aqueous methanol solution was used as a solvent. The methanol concentration in the pregel solution before addition of SF was 40 volume %. Therefore, the methanol concentration in the pregel solution after addition of SF was 8 volume %.

The gel obtained in this experiment was more flexible than the gels obtained in Reference Experiment 1-1 and Experiment 1-2, and magnifying observation under a microscope showed that coating with the gel was uniform. However, the needle part was deformed by gelation of the pregel solution.

### Reference Experiment 2-1

This experiment shares a common fundamental concept with Experiment 1-1, but the surface of the needle part is coated with a PBA gel by a second method. First, as in Reference Experiment 1-1, a molded body fabricated with SF was molded. Subsequently, before the molded body was removed from the mold, a pregel solution was injected between the mold and the molded body. As a pregel solution, the pregel solution of Prescription 1 used in Reference Experiment 1-1 was used. The pregel solution was injected by syringe injection. After injection of the pregel solution, the mold was placed in liquid paraffin for a liquid seal. Subsequently, the mold placed in the liquid paraffin was transferred to an oven at 60° C. and left overnight to polymerize the pregel solution. Then, the molded body was removed from the mold, and thereafter a microneedle was obtained in the same manner as in Reference Experiment 1-1.

When the obtained microneedle was observed under a microscope, only minimum coating was observed. This may be possibly because the pregel solution leaked from a gap between the molded body and the mold into the liquid paraffin while the mold was placed in the liquid paraffin.

### Reference Experiment 2-2

This experiment is different from Reference Experiment 2-1 in that a pregel solution of a different prescription was used, and the polymerization condition is therefore also different from that in Reference Experiment 2-1. First, as in Reference Experiment 2-1, a molded body fabricated with SF was molded using a mold, and then a pregel solution was injected between the mold and the molded body. As a pregel solution, the pregel solution of Prescription 2 used in Reference Experiment 1-2 was used. After the pregel solution was injected, the mold was left at room temperature for polymerization. After one hour, the molded body was removed from the mold, the removed molded body was washed with ultrapure water and dried to obtain a microneedle.

Observation of the obtained microneedle under a microscope showed that smooth coating of the needle part with a PBA gel could not be achieved.

### Reference Experiment 2-3

This experiment is different from Reference Experiment 2-1 in that a pregel solution of a different prescription was used, and the polymerization condition is therefore also different from that in Reference Experiment 2-1. Specifically, in this experiment, a microneedle was obtained in the same manner as in Reference Experiment 2-2 using the pregel solution of Prescription 3 used in Reference Experiment 1-3.

Observation of the obtained microneedle under a microscope showed smooth coating of the needle part with a PBA gel could not be achieved as in Reference Experiment 2-2.

### Reference Experiment 3

This experiment shares a common fundamental concept with Reference Experiment 1-1, but a needle part fabricated with SF and a PBA gel were combined by a method different from coating. First, an SF solution containing 20% by weight of polyethylene oxide (PEO) was poured into a mold and dried to obtain a molded body. The obtained molded body was removed from the mold and washed with ultrapure water to remove a soluble PEO portion from the molded body. By doing so, a molded body with a porous structure was obtained.

Subsequently, the molded body was immersed in a pregel solution to impregnate the porous structure of the molded body with the pregel solution. As a pregel solution, the pregel solution of Prescription 1 used in Reference Experiment 1-1 was used. Thereafter, a microneedle was obtained in the same manner as in Reference Experiment 1-1.

Observation of the obtained microneedle under a microscope showed that a needle part with a smooth surface was not obtained, and the mechanical strength of the needle part was markedly weak.

### Reference Experiment 4

In this experiment, a needle part fabricated with SF was obtained with a porous structure by a method different from Reference Experiment 3. First, an SF solution was poured into a mold and centrifuged to fill the SF solution into the tip of a cavity of the mold. Subsequently, the mold containing the SF solution was lyophilized using liquid nitrogen to obtain a molded body with a porous structure. Subsequently, the molded body was immersed in a pregel solution to impregnate the porous structure of the molded body with the pregel solution. As a pregel solution, the pregel solution of Prescription 2 used in Reference Experiment 1-2 was used. After the molded body was impregnated with the pregel solution, a microneedle was obtained in the same manner as in Reference Experiment 1-2.

Observation of the obtained microneedle under a microscope showed that the needle part was not formed in a favorable pyramidal shape. In particular, the tip portion, which is particularly important for insertion into the skin, was not formed appropriately.

### Reference Experiment 5-1

In this experiment, the fundamental concept is different from those of Reference Experiments 1 to 4 above, and the needle part was formed with a hybrid gel of a PBA gel and SF combined. First, as a pregel solution, a hybrid pregel solution was provided by further adding SF to the pregel solution of Prescription 2 used in Reference Experiment 1-2. The SF concentration of the hybrid pregel solution was 48% by weight.

The provided hybrid pregel solution was poured into a mold, the hybrid pregel solution was filled into a cavity portion corresponding to the tip of the needle part by centrifugation and dried for 4 to 6 hours. Polymerization occurs while the hybrid pregel solution is being dried. Pouring of the hybrid pregel solution into the mold, centrifugation, and drying were repeated several times to obtain a microneedle having a needle part and a base part fabricated with a hybrid gel.

Observation of the obtained microneedle under a microscope showed that the tip region of the needle part had been contracted. This may be possibly because crystallization of SF was caused by the high methanol concentration of the hybrid pregel solution used in this experiment.

### Reference Experiment 5-2

A microneedle was obtained in the same manner as in Reference Experiment 5-1, except that a hybrid pregel solution was provided by further adding SF to the pregel solution of Prescription 3 used in Reference Experiment 1-3.

Observation of the obtained microneedle under a microscope showed that the tip region of the needle part was prevented from being contracted by using a hybrid pregel solution with a lower methanol concentration compared with the hybrid pregel solution used in Reference Experiment 5-1. However, extreme deformation of the base part due to contraction of the base part during the step of drying was observed.

### Experiment 6 (Example)

This experiment is different from Reference Experiment 5-2 in that only a needle part is formed with a hybrid gel. First, as in Reference Experiment 5-2, a hybrid pregel solution was provided by further adding SF to the pregel solution of Prescription 3. Subsequently, as in Reference Experiment 5-1, pouring of the hybrid pregel solution into the mold, centrifugation, and drying (polymerization) were repeated multiple times to form a needle part fabricated with a hybrid gel. After forming the needle part, an SF solution was poured into a cavity portion corresponding to the base part of the mold including the needle part and dried. The obtained molded body was removed from the mold, washed with ultrapure water, and dried. By doing so, a microneedle was obtained with a double-layered structure of a needle part fabricated with a hybrid gel and a base part fabricated with SF.

No deformation was observed in the obtained microneedle because the base part was formed with SF. The tip of the needle part was formed in a sharp pyramidal shape.

In preparing the microneedle by Experiment 6, polymerizable acryloxyethyl thiocarbamoyl rhodamine B (polymerizable fluorescent monomer) was added to a hybrid pregel solution to check positions where the hydrogel was present. The results showed that the hydrogel was favorably present in the tip portion.

The results of the above-described experiments suggest that Experiment 6 is suitable as a method for producing the microneedle of the present invention. Additionally, a small volume of the hybrid pregel solution used in Experiment 6 is sufficient because the volume sufficient to constitute the needle part is required, rapid polymerization is possible at normal temperature, and the monomer concentration and the SF concentration can be suitably adjusted. The microneedle with a double-layered structure according to Experiment 6 enables formation of a sharp needle tip having susceptibility to glucose. This is very important for the release of insulin according to the blood glucose concentration in a self-regulated manner. Additionally, the microneedle according to Experiment 6 was stable in an atmosphere at 37° C. for at least 7 days, and no apparent morphological change was identified even by SEM observation.

### [Gel Swelling Test]

Because N-isopropylacrylamide (NIPAAm) is a temperature-sensitive material, it is important to assess swelling of the hydrogel at various temperatures and glucose concentrations. Changes in the volume were measured using the NIPAAm/4-(2-acrylamide ethylcarbamoyl)-3-fluorophenylboronic acid (FPBA) gel composition and the semi-interpenetrating network (semi-IPN) gel composition at various glucose concentrations and temperatures.

The NIPAAm/FPBA gel composition is a gel composition obtained by using a pregel solution of the following prescription:

Gelling agent: NIPAAm
Phenylboronic acid-based monomer: AmECFPBA
Crosslinking agent: MBAAm
Solvent: aqueous methanol solution
NIPAAm/AmECFPBA=92.5 mol/7.5 mol
Charging percent of crosslinking agent to the monomers=2%
Monomer concentration: 1.5 mol/L (Sample 7-1), 1 mol/L (Sample 7-2)

The semi-interpenetrating network gel composition is a gel composition obtained by obtaining a gel composition using a pregel solution obtained by adding SF to the pregel solution of the above-described prescription so that the weight fraction of SF based on the total monomer weight is 48% by weight and then further performing methanol treatment of immersing the gel composition in an aqueous methanol solution for 30 minutes. As semi-interpenetrating network gel compositions, a sample with a monomer concentration of 1.5 mol/L (Sample 7-3) and a sample of 1 mol/L (Sample 7-4) were obtained.

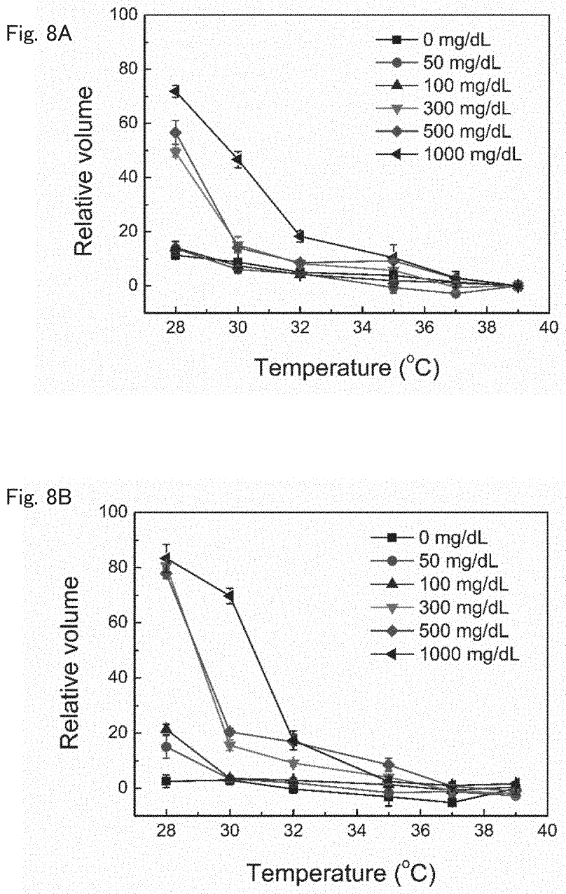

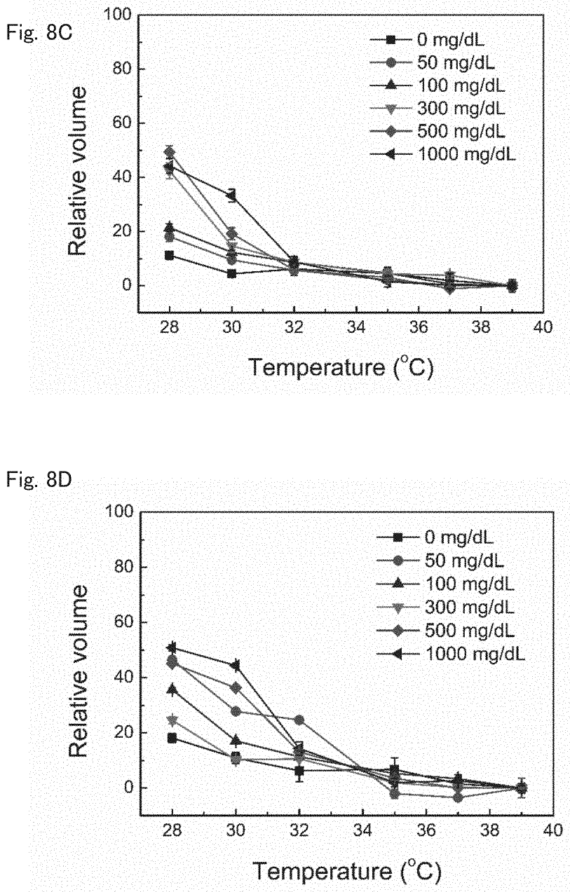

For these samples, relative volume changes were obtained by equilibrating with a PBS buffer solution (pH 7.4) at various glucose concentrations and temperatures for 24 hours and measuring the sample diameters on microscopic images. The results are shown in FIGS. 8A to 8D.

As shown in FIGS. 8A to 8D, only slight volume changes were observed in all the gel compositions in a range from skin temperature (32° C.) to physiological temperature (37° C.), with limited swelling and contraction at these temperatures. The volume changes of the NIPAAm/FPBA gel composition increased as the monomer concentration decreased from 1.5 mol/L (FIG. 8A) to 1 mol/L (FIG. 8B), but the increase was not so marked in the case of the semi-interpenetrating network gel composition containing SF (FIGS. 8C and 8D). This may be possibly because further crystallization due to incorporation of SF into a 3D polymer network prevents relaxation and mobility of polymer chains.

According to the free volume theory, the diffusion rate of a solute in a hydrogel depends on the mesh size in mobility of a specific polymer chain that has a certain polymer-solute interaction. Therefore, the solute diffusion rate usually decreases as the water volume percent in a gel composition decreases. Despite such a minimum swelling, an insulin release highly synchronized with the glucose concentration is achieved in all gel compositions containing boronic acid. It is considered that a threshold mesh size suitable for regulation of insulin diffusion was probably achieved in these gel compositions. As a result, sufficient regulation of insulin diffusion was achieved with hydration change at the threshold level in response to the glucose concentration. Further, electrostatic repulsive force between anionic insulin and a negatively charged boronic acid-glucose composite may promote a release of insulin. The present inventors' previous studies also showed similar phenomena.

Further, compared with the NIPAAm/FPBA gel compositions, a smaller volume change was observed in a semi-interpenetrating network gel composition that contained SF and was treated with an aqueous methanol solution. However, susceptibility to glucose was the same (Siyuan Chen et al., “Microneedle-Array Patch Fabricated with Enzyme-Free Polymeric Components Capable of On-Demand Insulin Delivery,” Advanced Functional Materials, issued on Dec. 9, 2018). This finding indicates that even after physical crosslinking of the SF components, a polymer gel network incorporated into the semi-interpenetrating network structure containing SF can still be hydrated in response to glucose. This characteristic is advantageous because the effect of swelling on the mechanical toughness of the gel composition is reduced while a glucose-responsive functional group is maintained.

### [Re-Hydration Test]

In production and administration of a microneedle, a gel composition is dried during storage and subsequently re-hydrated with an interstitial fluid after administration into the skin. It is therefore important to investigate the inner structure of the gel composition after re-hydration. The NIPAAm/FPBA gel composition and the semi-interpenetrating network gel composition (containing SF and treated with methanol) were prepared, dried at room temperature, and then re-hydrated with a PBS buffer solution (pH 7.4). After 24 hours, each gel composition sample was frozen in liquid nitrogen and then lyophilized. The lyophilized sample was carefully crushed to expose the inner structure. The sample was coated with gold, and a cross-sectional image was obtained using a scanning electron microscope (SEM).

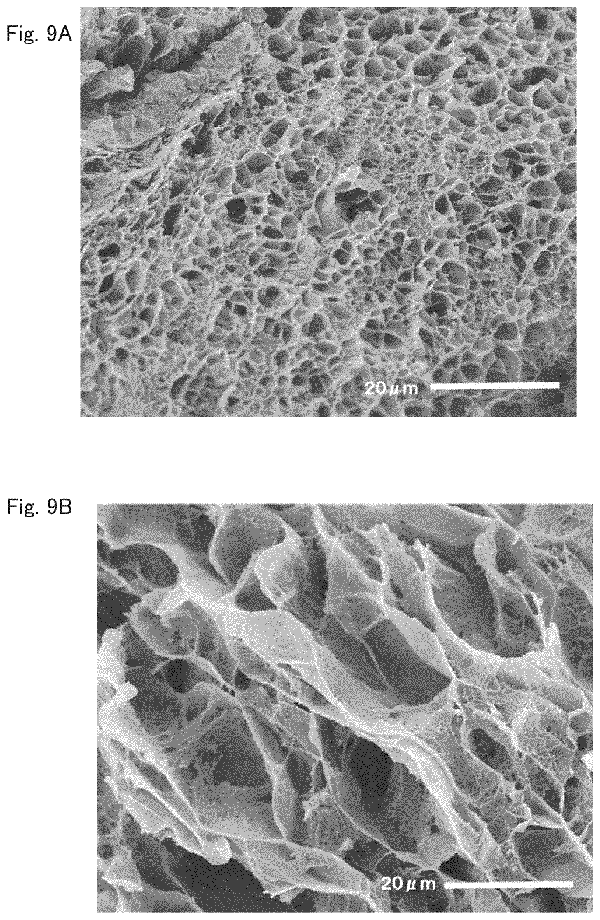

FIG. 9A shows a cross-sectional SEM image of the NIPAAm/FPBA gel composition, and FIG. 9B shows a cross-sectional SEM image of the semi-interpenetrating network gel composition. As shown in FIGS. 9A and 9B, both the gel compositions have an interconnected microporous structure, probably attributable to microscopic phase separation, which usually occurs during polymerization of a poly(acrylamide) derivative in a solvent (aqueous methanol solution). It is very important for drug delivery to maintain the interconnected microporous structure because it may promote diffusion of insulin and glucose filled in the matrix. Compared with the NIPAAm/FPBA gel composition (FIG. 9A), the semi-interpenetrating network gel composition treated with methanol and containing SF combined had a greater pore size and an enhanced surface roughness probably attributable to the presence of SF interdiffused in the polymer network and physical crosslinking of SF after methanol treatment (FIG. 9B).

### Other Embodiments of a Microneedle

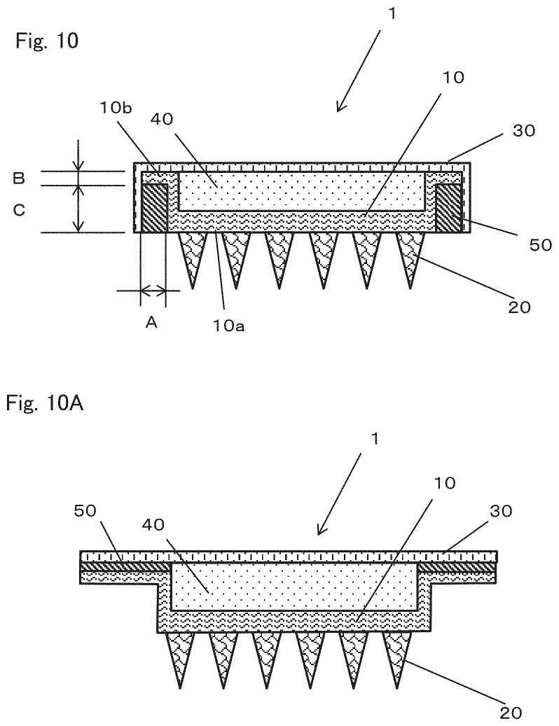

Leakage of insulin from the reservoir not through the base part is one of major problems for insulin delivery microneedles because it causes a burst release of insulin which may cause hypoglycemia. FIG. 10 is a schematic cross-sectional view showing an insulin delivery microneedle 1 that can prevent leakage of insulin not through the base part. Of note, in FIG. 10, the same reference signs as in FIG. 1 are imparted to components identical to or corresponding to those in FIG. 1, and unless otherwise specified, a configuration shown in FIG. 1 can be achieved with components having the same reference signs as in FIG. 1.

The insulin delivery microneedle 1 shown in FIG. 10 is the same as the one shown in FIG. 1 in that it has a flat, cup-shaped base part 10 having a recessed part used as an insulin reservoir 40 and a plurality of needle parts 20 provided on a bottom face 10a of the base part 10 and that the reservoir 40 is sealed with, for example, a silicone sheet 30 using an adhesive agent 50. However, the base part 10 is formed in a stepped shape having the flange 10a on the open-end side of the reservoir 40. The sheet 30 is hung toward the needle part 20 side over the flange 10a and also covers the base part 10 in the direction of the height of the base part 10. The adhesive agent 50 is applied over the whole circumference of the base part 10 between the base part 10 and the sheet 30 in the hung portion of the sheet 30.

According to such a structure, compared with the structure shown in FIG. 1, the sheet 30 can be bonded on the base part 10 in a greater bonded area and can seal the reservoir 40 more effectively. As a result, leakage of insulin from the reservoir 40 can be prevented effectively. In addition, swelling of the area of the insulin delivery microneedle 1 can be minimized.

The amount A of a flange 10b protruded can be set to, for example, 0.2 mm. Additionally, the thickness B of the flange 10b can be set to, for example, 0.1 mm, and the height C from the flange 10a to the bottom face of the base part 10 can be set to, for example, 0.2 mm.

In this embodiment, the planar shape of the insulin delivery microneedle 1 may also be an arbitrary shape such as a square or circular shape. Additionally, the outer shape of the flange 10a and the shape of the bottom face 10b of the base part 10 on which needle parts 20 are arranged may be identical to or different from each other. Both the outer shape of the flange 10a and the shape of the bottom face 10b are preferably circular in light of preventing deformation during production of the insulin delivery microneedle 1.

Additionally, if swelling of the area of the insulin delivery microneedle 1 is allowed, the bonded area can be increased by increasing the amount of the flange 10a protruded, and bonding a sheet 40 on the upper face of the flange 10a via the adhesive agent 50 as shown in FIG. 10A

### [In Vivo Evaluation]

To evaluate the glucose-responsive insulin release in vivo, a glucose tolerance test was performed in mice. The glucose tolerance test was performed using a phosphate-buffered saline (PBS)-filled microneedle and a Humulin (human insulin)-filled microneedle. The epidermis of four healthy mice was treated with the PBS-filled microneedle, and the epidermis of three healthy mice was treated with the Humulin-filled microneedle. As a microneedle, the structure shown in FIG. 1 was used.

On Day 2 of administration of the microneedles, glucose (2 g/kg) was injected to all mice after fasting for 2 hours. Blood glucose concentrations (blood sugar levels) were measured with a blood glucose meter at 0, 30, 60, and 90 minutes after glucose injection. At these time points, blood was also drawn from the caudal vein of mice and centrifuged at 2000 g for 15 minutes to collect serum. Humulin present in serum was analyzed using an insulin ELISA kit.

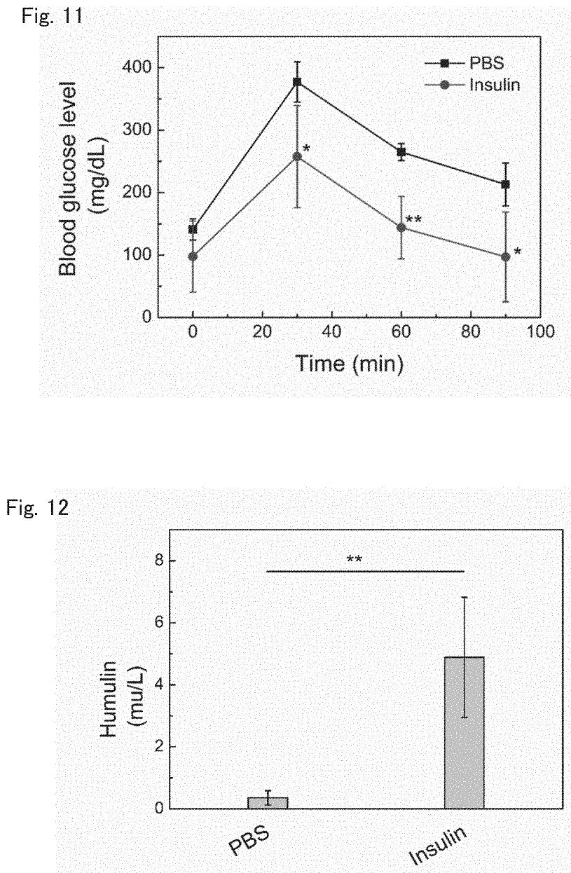

FIG. 11 is a graph showing changes with time in blood sugar levels. As shown in FIG. 11, blood sugar levels in mice treated with the PBS-filled microneedle (also referred hereinafter to as the PBS group) increased markedly 30 minutes after glucose injection. However, the increases in the blood sugar level in mice treated with the Humulin-filled microneedle (also referred hereinafter to as the Humulin group) were not marked compared with the PBS group, because a marked amount of Humulin was released into blood (see FIG. 12). The difference between these two groups was more marked at 60 minutes after glucose injection. The blood sugar level after 90 minutes returned to the pre-treatment value in the Humulin group but was approximately 250 mg/dL in the PBS group. These data show the glucose responsiveness of the microneedle in the Humulin group.

Of note, whether the difference between the PBS group and the Humulin group was statistically significant was tested by Student's t test. In FIGS. 11 and 12, “*” indicates that the p value is lower than 0.05 in the Humulin group in comparison with the PBS group, and “**” indicates the p value is lower than 0.01 in the Humulin group in comparison with the PBS group.

### EXPLANATION OF SYMBOLS

1 insulin delivery microneedle
10 base part
20 needle part
30 sheet
40 reservoir
50 adhesive agent
100 mold
101 cavity

1. A glucose-responsive composite gel composition comprising: a copolymer comprising a phenylboronic acid-based monomer unit; and silk fibroin.

2. The glucose-responsive composite gel composition according to claim 1, wherein a proportion of the silk fibroin in a solid content of the composite gel composition is 10 to 90% by weight.

3. The glucose-responsive composite gel composition according to claim 1, wherein the phenylboronic acid-based monomer unit comprises a phenylboronic acid-based monomer, a gelling agent, and a crosslinking agent.

4. A method for producing a glucose-responsive composite gel composition, comprising:
providing a monomer mixture comprising a phenylboronic acid-based monomer; and
copolymerizing the monomer mixture in a presence of silk fibroin.

5. The method for producing a glucose-responsive composite gel composition according to claim 4, wherein a proportion of the silk fibroin in a total solid content is 10 to 90% by weight in the step of copolymerizing.

6. The method for producing a glucose-responsive composite gel composition according to claim 4, wherein the copolymerizing is performed at normal temperature.

7. The method for producing a glucose-responsive composite gel composition according to claim 4, wherein the monomer mixture comprises the phenylboronic acid-based monomer, a gelling agent, a crosslinking agent, and a solvent.

8. The method for producing a glucose-responsive composite gel composition according to claim 7, wherein the solvent contains methanol.

9. An insulin delivery microneedle comprising:
a base part;
at least one needle part integrally provided on the base part; and
an insulin reservoir, wherein
the base part has a mechanical strength required to support the needle part and is fabricated with a material having insulin permeability, and
at least a tip portion of the needle part comprises a glucose-responsive composite gel composition according to claim 1.

10. The insulin delivery microneedle according to claim 9, further comprising a recessed part formed in the base part and a sheet covering the recessed part, wherein the reservoir is formed with a sealed space between the base part and the sheet.

11. A method for producing an insulin delivery microneedle comprising a base part and at least one needle part integrally provided on the base part, the method comprising:
providing a mold in which a cavity corresponding to the base part and the needle part is formed;
injecting a pregel solution containing a monomer mixture comprising a phenylboronic acid-based monomer and silk fibroin into a cavity portion corresponding to the needle part in the mold;
polymerizing the monomer mixture in the pregel solution to form a composite gel composition containing the silk fibroin;
forming the base part having a mechanical strength required to support the needle part and fabricated with a material having insulin permeability, in a cavity portion corresponding to the base part in the mold containing the composite gel composition, integrally with the composite gel composition; and
removing a molded body which is obtained from the mold after the base part is formed.
